(19) United States
(12) Patent Application Publication
(10) Pub. No.: US 2022/0287840 A1
(43) Pub. Date: Sep. 15, 2022

(54) A MEDICAL ARRANGEMENT FOR INTRODUCING AN OBJECT INTO AN ANATOMICAL TARGET POSITION

(71) Applicant: Medtentia International Ltd Oy, Espoo (FI)

(72) Inventor: Olli Keränen, Bjärred (SE)

(21) Appl. No.: 17/625,069

(22) PCT Filed: Jul. 3, 2020
(86) PCT No.: PCT/EP2020/068868

§ 371 Date: Jan. 5, 2022

(30) Foreign Application Priority Data
Jul. 8, 2019 (EP) 19184993.4

(51) Int. Cl. A61F 2/24

(52) U.S. Cl. CPC A61F 2/2466; A61F 2230/0041; A61F 2230/0091; A61M 2025/0681

(57) ABSTRACT

A medical arrangement for introducing an implant into an anatomical target position comprises a first introducer and a guide wire. The guide wire is configured to be introduced before the implant into or towards the anatomical target position. The implant is configured to be delivered along the guide wire into the anatomical target position. The guide wire is configured to be retracted after the implant has been introduced into said anatomical target position so that said object essentially maintains the shape taken when introduced into said anatomical target position.

### TECHNICAL FIELD OF THE INVENTION

The invention relates to a medical arrangement for introducing an object, such as an implant into an anatomical target position, such as a cardiac implant (like an annuloplasty medical device) into an annulus of a heart valve, such as a mitrel valve or tricuspid valve.

### BACKGROUND OF THE INVENTION

FIG. 1A illustrates a portion of the heart 12, the mitral valve 18, and the left ventricle 14 as an example of the anatomical target position. The mitrel valve is at its boundary circumferenced by an annulus 20. The valve has two cusps or leaflets 22, 24. Each of these cusps or leaflets 22, 24 are connected to a respective papillary muscle 27, 29 via their respective connecting chordae 26, 28. In normal healthy individuals the free edges of the opposing leaflets will close the valve by coaptation. However, for some individuals the closure is not complete, which results in a regurgitation, also called valvular insufficiency, i.e. back flow of blood to the left atrium making the heart less effective and with potentially severe consequences for the patient. FIG. 1B illustrates a mitral valve 18, in which the leaflets 22, 24 do not close properly. This commonly occurs when the annulus 20 becomes dilated. One surgical procedure to correct this is to remove a portion of the leaflet 24 and stitch the cut edges together with one another. The procedure will pull back the annulus 20 to a more normal position. However the strength of the leaflet 24 is altered. Similar problems with a less effective heart function occur if one or both leaflets are perforated to such an extent that blood is flowing towards the left atrium, although the leaflets close properly.

In some conditions of degenerated heart function, the leaflets do not present a solid surface, as in a degenerative valve disease. The leaflet may also be ruptured, most commonly at an edge of a leaflet, resulting in an incomplete coaptation. Hence, cardiac devices and methods are developed for repairing of one or more leaflets of a heart valve, or other related anatomical structures, such as the chordae attached to the ventricular side of leaflets.

FIG. 2 illustrates an exemplary implant to be delivered and introduced into an anatomical target position, and in particularly a cardiac implant 110. The implant may comprise one or more loop-shaped structures 111, 112. Advantageously one first loop-shaped structure is configured to abut a first side of the heart valve and one second loop-shaped structure is configured to abut a second, opposite, side of the valve to thereby trap a portion of the valve tissue 20 between the second and the first support structures 111, 112.

The implant is typically delivered via a catheter and has thus typically a delivery state, where the implant has an elongated form. In said delivery state the implant can be transferred advantageously through a catheter having diameter 7-10 mm, for example. The implant comprises typically a shape memory material having a first shape, such as the elongated form of the delivery state in a first temperature, and the second shape, such as the loop-shaped form in a second temperature. The second temperature corresponds advantageously essentially to the body temperature, whereupon the implant takes the second shape, corresponding to the loop-shaped form, when introduced for example with the blood flow in the atrium.

In addition, some problems arise due to a catheter system having both inner and outer catheters, usually steerable catheters, sometimes numerous inner catheters, where the inner catheter(s) locating inside the outer catheter limit(s) space from the implant.

It is found that the prior art cardiac implants, such as depicted above, work very well, but there are still some disadvantages relating to the introduction devices, such as catheter type devices, to deliver the implant into the anatomical target position, such as into the annulus of the heart valve. The catheter based systems are based for delivering a relatively thick main catheter having a first curve portion into a first portion of the anatomical target position, such as to an atrium, then a second catheter having a second curve portion into a second portion of the anatomical target position, such as next to annulus or leaflets of the heart valve, and then a third catheter having a third curve portion into a third portion of the anatomical target position, such as around the annulus of the heart valve. In some systems there might be even further catheters with further curve portions to be delivered before the implant can be delivered and introduced into its position. The third or further catheter if used, is called as a delivery catheter. The implant is then delivered to its position inside the feeding catheters, which is delivered to its position inside the other catheters.

There are some drawbacks related to the prior art catheter based systems, such as at least the main catheter must be thick (7-10 mm or even more) so that it can carry the further catheters inside. In addition, also the delivery catheter must be relatively thick so that the implant can be delivered inside the delivery catheter. When the catheters are relatively thick, it is very hard to insert the catheters into the anatomical target position. For example, a sub-annular space below the annulus, so between the chordae and wall or septum, is very narrow, whereupon the best channel for the thick catheters is very difficult to find and deliver, in particular when the maneuverability and steerability of the catheters is poor. Furthermore, the surface of the inner wall is rough, having additionally numerous attachment points of chordae, which easily causes stuck of the catheters. Additional challenges arise when the catheters, especially also the delivery catheter, have memory properties or predetermined shapes, which might activate too early and thereby raising possibility to stuck the catheter into the wall or other structures of the anatomical target position, such as the heart. Thus, the time limit to insert the catheters, in particularly the delivery catheter, having memory properties, is very limited so that the catheters can be inserted into their right and accurate position before the memory property will be activated by the temperature of the anatomical target position, such as the heart.

### SUMMARY OF THE INVENTION

It is an object of the invention to alleviate and eliminate the problems relating to the known prior art. Especially the object of the invention is to provide a medical arrangement for introducing an implant into an anatomical target position in an easy, fast, safe and accurate manner with a high degree of control. In addition, the object of the invention is to minimize the sizes of the catheters used and at the same time minimize stuck of the catheters and thereby minimize stress introduced for the anatomical target position.

The object of the invention can be achieved by the features of independent claim.

The invention relates to a medical arrangement for introducing an implant into an anatomical target position, such as a cardiac implant into an annulus of a heart valve, according to claim 1.

A medical arrangement according to the invention is configured to introduce an object, such as an implant, and in particularly such as a cardiac implant, or an annuloplasty medical device, from a distal end of the arrangement into an anatomical target position, such as into an annulus of a heart valve. The heart valve may be a mitrel valve or tricuspid valve, for example, not limiting to those only. It is to be understood that the object can be also some other object, such as medicine, for example.

According to an example the object is the implant, which comprises in a use a loop shaped support portion, having either one or more loops or coils so that at least one first loop-shaped structure can be configured to abut a first side of the heart valve and at least one second loop-shaped structure to abut a second, opposite, side of the valve to thereby trap a portion of the valve tissue between the second and the first support structures. It is also possible that there is only the one first loop-shaped structure, which is configured to abut a first side of the heart valve, and not the second support structures, or vice versa. The implant is advantageously adapted to support a mitrel valve upon being fully delivered.

According to an embodiment the medical arrangement comprises a first introducer, such as an outer steerable introducer or catheter, and a guide wire. The first introducer is advantageously an outer steerable catheter, which is delivered for example to the atrium or ventricle, but not to the anatomical target end position as such, where the implant is to be introduced. According to an embodiment the guide wire is configured to be introduced into or towards said anatomical target position via the first introducer but before the implant (meaning that the guide wire is delivered beforehand so that it can guide the delivery of implant). When the guide wire is delivered into the right position, the implant is then delivered along and guided by the guide wire (and inside the first introducer) into the anatomical target position.

The guide wire can be also introduced partly from the distal end portion of the first introducer, after which also the implant can be delivered partly along the guide wire (i.e. not to a final end target position at once but rather step by step), and after which the guide wire may be further delivered and followed by the implant and thereby introducing the guide wire and implant sequentially. According to an embodiment the guide wire and implant can be delivered at the same time, but however so that the guide wire guides the route to the anatomical target position.

The implant is advantageously a hollow tubular structure, whereupon the implant can be delivered into said anatomical target position so that it travels around or over the guide wire. This offers clear advantages over the known prior art solutions namely because the guide wire is very thin compared to the catheters, it is remarkable easy, fast, safe and accurate to insert via very small and narrow channels of the anatomical target position to the correct place. When the guide wire is delivered to the correct position, the implant with the hollow tubular structure can be easily delivered along the guide wire to the correct end position. Alternatively, the implant may also have loops, stitches or turns coupled with it whereupon the implant, even a solid implant, can be delivered to the position so that the loops or turns travel around the guide wire. In addition, it is to be noted that the implant can be also made from fabric, polyurethane or polyester and there is no need for the implant to have any memory property. The implant can be delivered to the position for example by a pusher coupled to the proximal end of the implant, whereupon the implant can be pushed to the position by the pusher. In the case of a non-rigid implant, for example if the implant is made of textile or the like compressible material, the pusher can be coupled to the distal end of the implant and thereby to draw the implant to the position.

According to an embodiment the implant may have at least a first curved shape having a preformed shape and capable of being delivered in a straightened configuration through the first introducer, whereupon the implant is activated of guided to at least a first curved shape within or near the anatomical target position, such as in the atrium or ventricle proximate the mitral valve of the heart. The implant may comprise e.g. a shape memory property and biased to said curved shape. However, it is to be noted that according to the invention, in particularly the implants without any memory property can be delivered and guided by the guide wire, which is delivered to the position first.

It is to be noted that the implant is configured to follow said guide wire and said first curved shape of the guide wire into said anatomical target position. When the implant has been introduced into said anatomical target position, the guide wire can be retracted and the implant essentially maintains the shape taken when introduced into said anatomical target position, corresponding to the shape of the guide wire in the anatomical target position. It is to be noted that the implant can be attached, such as sutured, into its position for example by keeping the implant in its position during the attaching by the guide wire. The implant can also be a self-attaching version having e.g. teeth, whereupon when the introducer or catheter over the implant is retracted, the teeth can dig into the tissue and thereby attach the implant into its position.

The guide wire is advantageously retracted only after the implant is secured. This makes the attaching of the implant very safe because the implant can be retracted away as long as the guide wire is in the anatomical target position. In addition it is to be noted that when the guide wire is inside the implant and in the anatomical target position, the position of the implant can still be adjusted by manipulating the guide wire, such as by dragging and/or turning the guide wire and thereby adjusting the position or orientation of the implant (a whole packet of the guide wire and the implant), which is very advantageous in many situations. In addition, if something goes wrong, the implant can still be retracted away by the guide wire. After the implant is attached to the anatomical target position the guide wire can be retracted away from the anatomical target position.

The guide wire has advantageously an activated shape and an inactivated shape, wherein in said inactivated shape the guide wire can be delivered in a straightened configuration through the first introducer and in said activated shape the guide wire takes at least a first curved shape within or near the anatomical target position, such as in the atrium or ventricle or proximate the mitral valve. The guide wire advantageously has a preformed shape so to take the activated shape when introduced to the anatomical target position. According to an embodiment the guide wire is at least partially formed from a shape memory material and thereby operable to assume said activated shape when meeting the temperature of the anatomical target position. The activated shape can be achieved also via another techniques known by the skilled person. When the guide wire is activated to said at least first curved shape and delivered to the anatomical target position (or at least towards the position), after which the implant is delivered along the guide wire to the anatomical target position, as is described elsewhere in this document.

In addition, the distal end of the guide wire may comprise a curvature tip portion, such as a J-shape, in order to allow smooth delivery of the distal end of the guide wire and to prevent the distal end of the guide wire from getting tangled into tissue. The curvature tip portion may have a preformed shape or being at least partially formed from a shape memory material and thereby taking the shape of the curvature tip portion when introduced into the anatomical target position.

According to an embodiment the arrangement may additionally comprise a second introducer, such as a catheter and in particular an inner steerable catheter. The second introducer is arranged to be operable between the first introducer and the guide wire. The second introducer is configured to be introduced from the distal end portion of the first introducer. According to an embodiment, the second introducer is an optional introducer and can be used for example to bypass the leaflets of the heart or other anatomical portion in or near the anatomical target position. However, according to an embodiment at least a distal portion of the second introducer is configured to be introduced from the distal end portion of the first introducer before the guide wire, and thereby guide or instruct the guide wire to bypass for example a certain anatomical portion, such as leaflets.

If the second introducer is used, the implant may then be delivered inside the second introducer (and also inside the first introducer), and again along the guide wire, into or towards the anatomical target position after at least the distal portion of the second introducer is introduced from the distal end portion of the first introducer.

However, the second introducer is advantageously configured to be retracted after said guide wire is introduced into said anatomical target position and/or before said implant is introduced into said anatomical target position. This is not mandatory but by this more space can be provided for the implant.

In addition, according to an embodiment, the arrangement may additionally comprise also a guiding catheter, which could also be called a third introducer, where said guiding catheter is arranged to be operable between the first introducer and the guide wire, and if the second introducer is used, also between the second introducer and the guide wire. The guiding catheter can be used as a delivering catheter for delivering the implant into the anatomical target position inside the guiding catheter but still around the guide wire. When the implant is delivered inside the guiding catheter, it additionally protects the anatomical portions from the implant. The guiding catheter is configured to be introduced from the distal end portion of the first introducer (and from the distal end portion of the second introducer, if used). It is to be noted that at least a distal portion of the guiding catheter is introduced from the distal end portion of the first introducer (and from the distal end portion of the second introducer, if used) only after the guide wire is introduced into or towards said anatomical target position.

The guiding catheter is advantageously a flexible catheter, which is configured to be delivered along the guide wire into or towards said anatomical target position after the guide wire is at least partially introduced from the distal end portion of the first introducer. Most advantageously the guiding catheter is delivered to the anatomical target position when the guide wire is fully delivered to the anatomical target position, in particular when the guiding catheter is flexible. The guiding catheter can be used to protect the tissue and other anatomical portions in the anatomical target position from the implant during delivering the implant into the anatomical target position. It is to be noted that the implant is configured to be delivered inside the guiding catheter into or towards said anatomical target position. This can be done after at least the distal portion of the guiding catheter is introduced from the distal end portion of the first and second introducers into or towards said anatomical target position, but most advantageously when the distal end of the guiding catheter is delivered into the anatomical target position.

According to an embodiment the second introducer is retraced before the implant is delivered into the anatomical target position inside the guiding catheter. This is not mandatory but by this more space can be provided for the implant, and especially when the guiding catheter is flexible so that it can expand for the implant during delivering. In addition, the guiding catheter is configured to be retracted after the guide wire and implant have been introduced into said anatomical target position.

Still in addition, the arrangement may also comprise a cooling arrangement for cooling the guide wire for example during retracting the guide wire, but also in other phases. When the guide wire is cooled down it can be easily reshaped and for example retracted out essentially easily transformable state. The cooling arrangement may be arranged so that there is an inlet for a cooling agent, such as cool water, in the proximal end of the first introducer, second introducer and/or guiding catheter, whereupon the cooling agent can flow between the walls of the first introducer, second introducer and/or guiding catheter from the proximal ends towards the distal ends, for example. According to an embodiment the distal ends may have openings so that the cooling agent can flow out.

The present invention offers advantages over the known prior art, such as an easy, safe, precise and time saving manner to reliable delivering the object, such as an implant to the anatomical target position, like to the annulus of the valve. Still, the guide wire is very convenient to deliver to the target position, namely it is very thin compared to the catheters and thus it does not tangle to the tissue. When the guide wire is delivered into the target position, the subsequent catheters, even flexible catheters, can be delivered easily along and guided by the guide wire. Further, the guide wire can be used as a rescue device during securing the object, like the implant, namely if something goes wrong in securing, any device used for pulling out the implant can be delivered to the proximal end of the implant along and guided by the guide wire, because the guide wire can be kept in the target position as long as the implant is secured.

In addition, the present invention provides for a compact arrangement for delivering the object. The compact medical device allows minimally invasive procedure. Furthermore, when using the compact catheter-operated medical device, risks for having any medical drawbacks or symptoms are much lower than e.g. in the traditional open-heart operation. Also, the patient recovery process is much faster.

The exemplary embodiments presented in this text are not to be interpreted to pose limitations to the applicability of the appended claims. The verb “to comprise” is used in this text as an open limitation that does not exclude the existence of also un-recited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objects and advantages thereof, will be best understood from the following description of specific example embodiments when read in connection with the accompanying drawings.

### BRIEF DESCRIPTION OF THE DRAWINGS

Next the invention will be described in greater detail with reference to exemplary embodiments in accordance with the accompanying drawings, in which:

FIGS. 1A-1B illustrate schematically a portion of a heart and mitral valve,

FIG. 2 illustrates an exemplary tissue anchoring unit for securing the flexible leaflet to the adjacent tissue and/or annulus according to an advantageous embodiment of the invention, and

FIGS. 3-23 illustrate a medical arrangement for introducing an implant into an anatomical target position according to advantageous embodiments of the invention.

### DETAILED DESCRIPTION

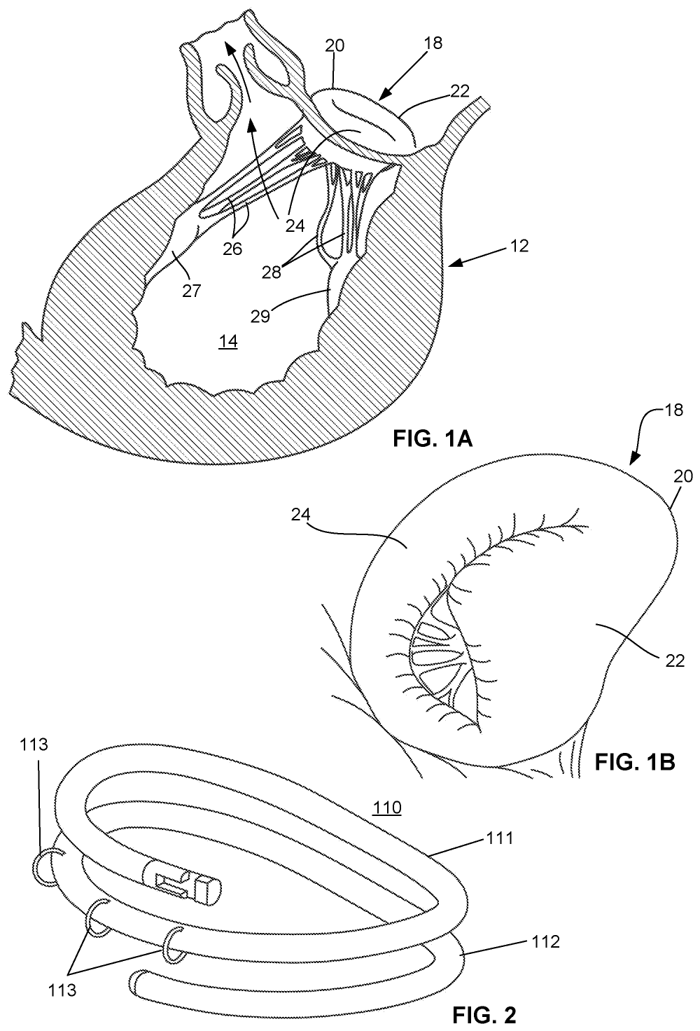

FIGS. 1A-1B and 2 are already discussed in more details in connection with the background of the invention portion above.

In addition, it is to be noted that the implant 110 comprises advantageously a hollow structure so that it can be passed along the guide wire 103 so that the guide wire 103 is inside the hollow structured implant 110. However, the implant 110 may also have loops, stitches or turns 113 coupled with it, as can be seen in FIG. 2, whereupon the implant, even a solid implant, can be delivered to the position so that the loops or turns 113 travel around the guide wire 103.

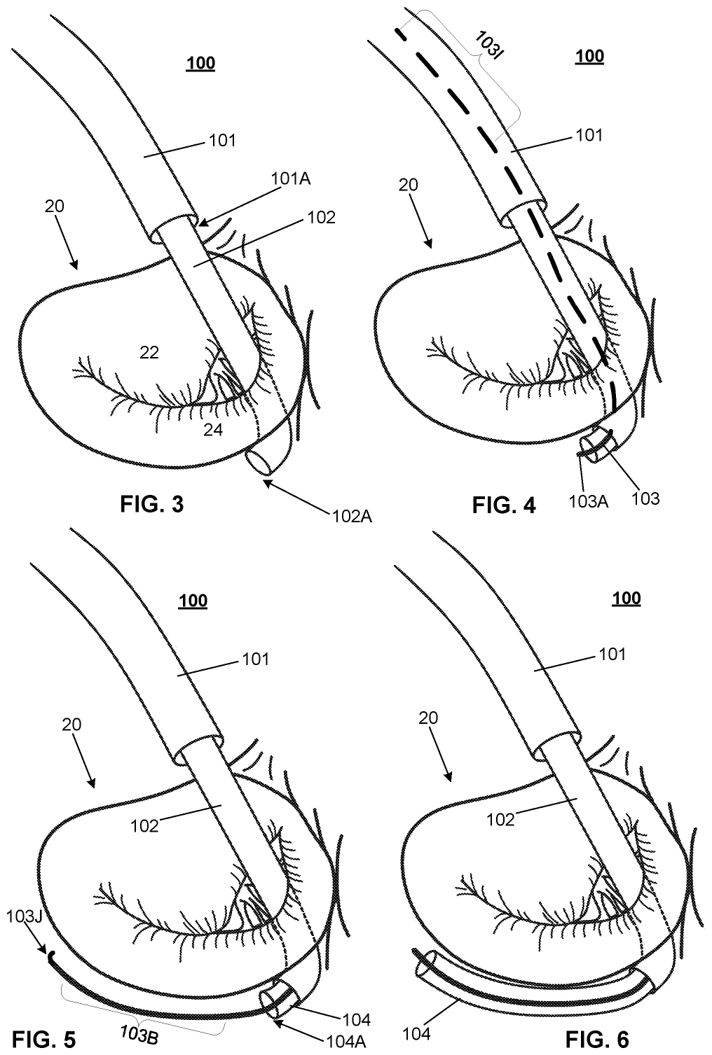

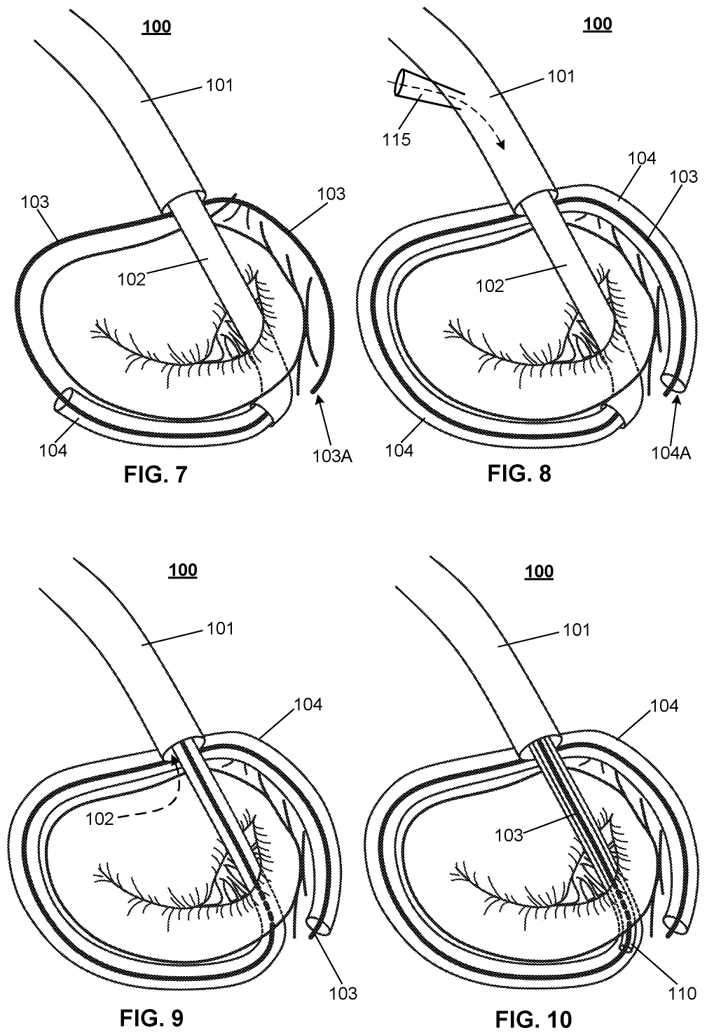

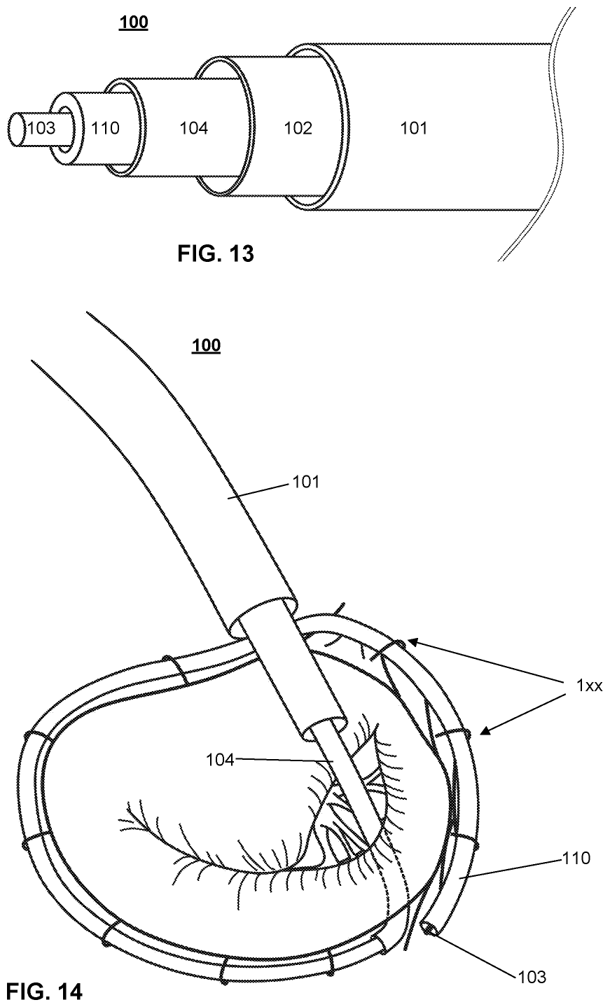

FIGS. 3-23 illustrate a medical arrangement 100 for introducing an implant into a heart as an example of the anatomical target position 20 according to advantageous embodiments of the invention. In FIG. 3 the first introducer 101 is delivered next to the annulus 20 of the heart, and the second introducer 102 is introduced from the distal end portion 101A of the first introducer 101. It is to be noted that the second introducer is an optional introducer, but it is still illustrated in Figures as an example. The second introducer can be used for example to bypass the leaflets 22, 24 of the heart, as well as to achieve a turn to a specific direction by turning the distal end 102A of the second introducer 102 (namely turnability of the introducers are limited). In addition, it is to be noted that at least a distal portion 102A of the second introducer 102 is introduced from the distal end portion 101A of the first introducer 101 before the guide wire, and may thereby guide or instruct the guide wire 103 to bypass the leaflets, for example, as can be seen in FIG. 4.

When the second introducer has bypassed the leaflets 22, 24, the guide wire 103 can be delivered into or towards the position 20. It is to be noted that the guide wire 103 is delivered into the position before the implant 110.

The guide wire 103 has advantageously an activated shape and an inactivated shape, wherein in said inactivated shape the guide wire 103 can be delivered in a straightened configuration 1031 through the first introducer 101, as can be seen in FIG. 4. In said activated shape the guide wire 103 takes at least a first curved shape 103B within or near the anatomical target position 20. When the guide wire 103 is activated to said at least first curved shape 103B, it is delivered to or at least towards the position. According to an example the distal end 103A of the guide wire 103 may comprise a curvature tip portion 103J, such as a J-shape, in order to allowing smooth delivery of the distal end 103A of the guide wire 103 and to prevent the distal end 103A of the guide wire 103 from getting tangled into tissue.

The arrangement may additionally comprise also a guiding catheter 104, as can be seen in FIGS. 5-11, where the guiding catheter 104 is operated between the first introducer 101 and the guide wire 103, and if the second introducer is used, also between the second introducer 102 and the guide wire 103. The guiding catheter 104 is introduced from the distal end portion 101A of the first introducer 101 (and from the distal end portion 102A of the second introducer 102, if used). It is to be noted that at least a distal portion 104A of the guiding catheter 104 is introduced from the distal end portion 101A of the first introducer 101 (and from the distal end portion 102A of the second introducer 102, if used) only after the guide wire 103 is introduced into or towards said anatomical target position 20.

The guiding catheter 104 is delivered along the guide wire 103 into or towards said anatomical target position 20 after the guide wire 103 is at least partially introduced from the distal end portion 101A of the first introducer 101, as can be seen in FIGS. 5-8. Most advantageously the guiding catheter 104 is delivered to the anatomical target position 20 when the guide wire is fully delivered to the anatomical target position, in particular when the guiding catheter 104 is flexible, whereupon the guiding catheter 104 is guided by the guide wire 103.

When the guide wire 103 and the guiding catheter 104 are delivered into the position, the second introducer 102 is retracted and it is retracted advantageously before delivering the implant 110 into the anatomical target position 20, as is the case in FIG. 9. This is not mandatory but by this more space can be arranged for the implant. It is to be noted that also the first introducer can be retracted before delivering the implant 110 or even right after the delivering of the second introducers, even if this is not shown in the Figures. By this even more space can be arranged for the implant.

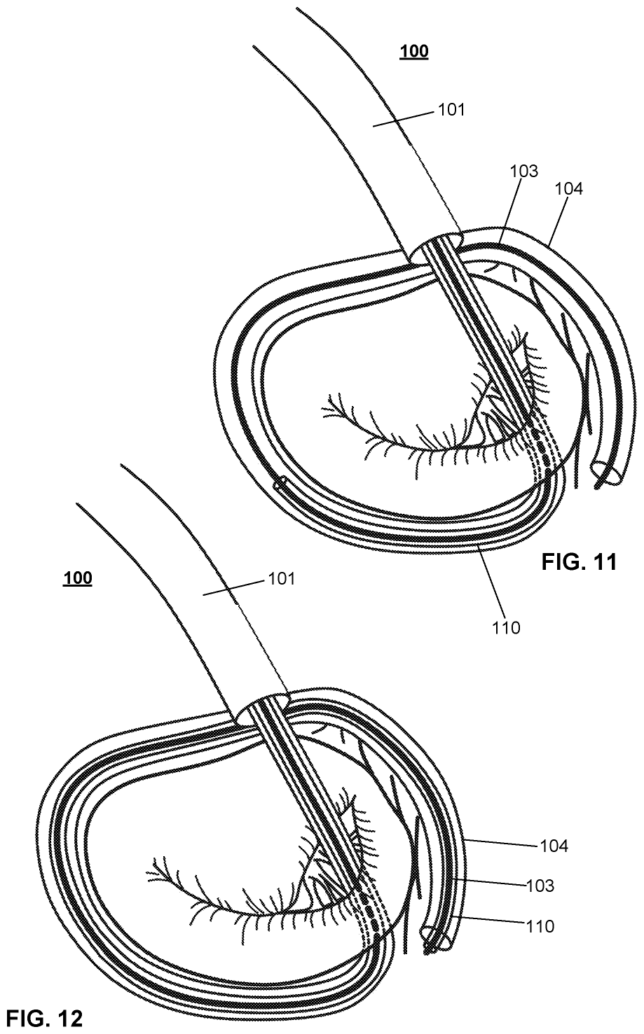

The implant 110 is then delivered around (and guided by) the guide wire 103 and inside the guiding catheter 104 into or towards said anatomical target position, as can be seen in FIGS. 10-12. This can be done after at least the distal portion 104A of the guiding catheter 104 is introduced from the distal end portion 101A of the first and second introducers 101, 102 into or towards said anatomical target position, but most advantageously when the distal end of the guiding catheter 104 is delivered into the anatomical target position.

The guiding catheter 104 is then retracted after the implant 110 is introduced into the anatomical target position 20 after which the implant can be secured to the tissue by securing members 114, as can be seen in FIG. 14. The securing can be done for example by suturing or stabling or by other securing methods known by the skilled person. In addition, the guiding catheter 104 can be retracted sequentially, uncovering only a part of the implant for securing, and after securing said uncovered part the guiding catheter 104 can be retracted more, thereby uncovering an additional portion of the implant for securing.

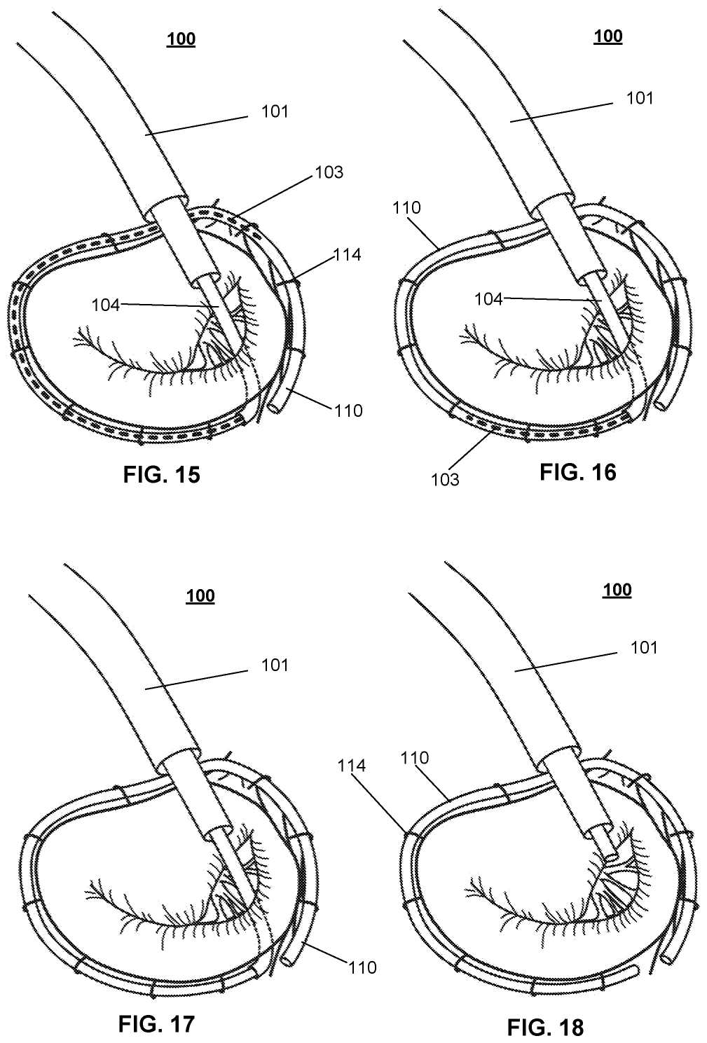

When the implant 110 is introduced and secured into the position 20 (and also the guiding catheter is retracted), as is the case in FIG. 10, the guide wire 103 can be retracted and the implant 110 essentially maintains the shape taken when introduced into said anatomical target position 20, corresponding to the shape of the guide wire in the anatomical target position, as can be seen in FIGS. 15-18.

FIG. 13 illustrates the order of the introducers 101 and 102, guiding catheter 104, guide wire 103 and the implant 110.

In addition, FIG. 8 illustrates also a cooling arrangement 115 for supplying a cooling agent (arrow) and thereby cooling the guide wire 103. It is to be noted that the arrangement may comprise one or more cooling arrangements 115 and arranged in connection with the first introducer, second introducer and/or guiding catheter. In addition, even if the cooling arrangement 115 is illustrated only in connection with FIG. 8, it should be understood, that it may also be comprised by other embodiments and arrangements 100 illustrated in other Figures.

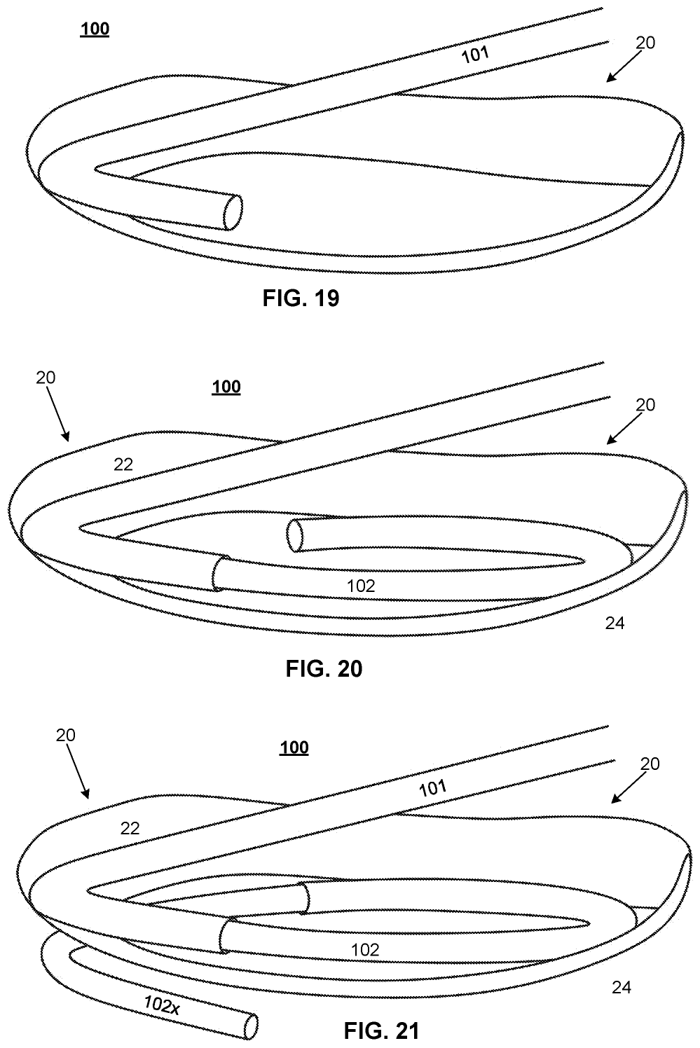

FIGS. 19-23 illustrate a further example of the medical arrangement 100 to introduce the implant 110 into a mitral valve 20 (as an example of the anatomical target position) so that at least one loop-shaped structure 111 of the implant 110 abuts a first side of the heart valve and one second loop-shaped structure 112 abuts a second, opposite, side of the valve to thereby trap a portion of the valve tissue 20 between the second and the first support structures 111, 112. In FIGS. 19-23 the first introducer 110 has a first curve shape and the second introducer 102 has a second curve shape to the same curvature direction as the first curve shape of the first introducer 101 so to form a concentric system 100. As can be seen in FIG. 21, the arrangement may also have an additional second introducer 102x, which again has third curve shape but still to the same curvature direction as the first and second curve shapes of the first and second introducers, wherein said first, second and third curved shapes are concentric curved shapes. In addition, the additional second introducer 102x may also be a steerable catheter or having pre-curved structure so that it has ability to seek said third curve shape at least and advantageously when delivered into or towards the anatomical target position.

It is to be noted that the first introducer is introduced next to the annulus and the second as well as additional second introducers 102, 102x are used to bypass the leaflets 22, 24 and to be introduced to the opposite side of the annulus as the first introducer is delivered.

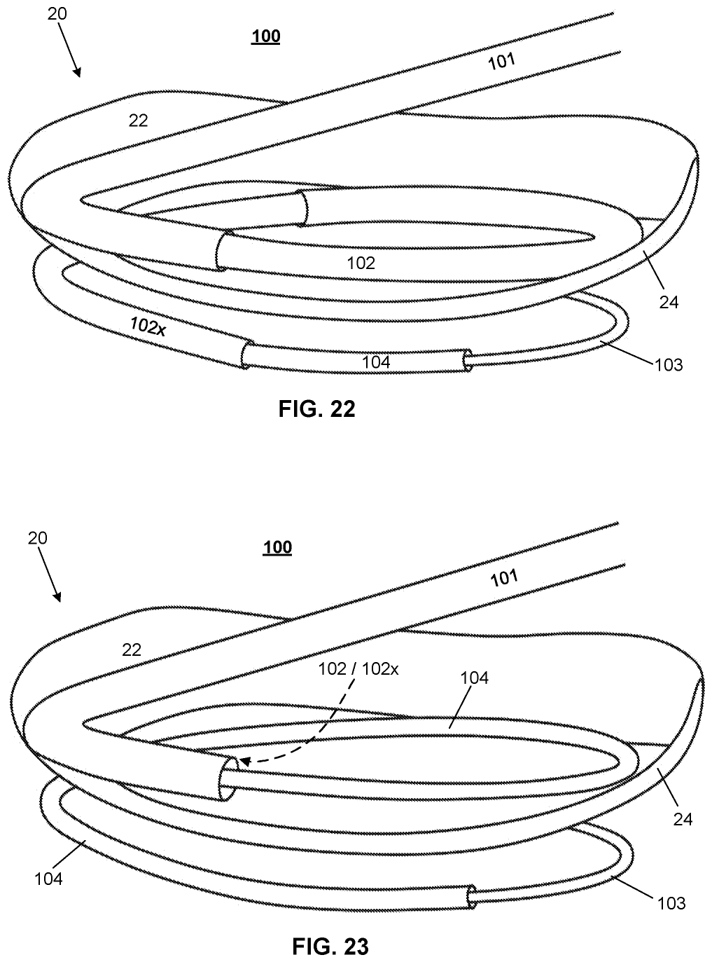

FIGS. 22 and 23 illustrate the arrangement 100, where the guiding catheter 104 and also the guide wire 103 are introduced into or at least towards the position for delivering the implant. As can be seen in FIG. 23, the second as well as additional second introducers 102, 102x can be retracted before introducing the implant. The implant and the steps for delivering it are not shown, but the fundamental principles are the same as described elsewhere in this document.

It is to be noted that according to an embodiment the first introducer 101 can be retracted already after the second introducer 102 is delivered and before the delivery of the additional second introducer 102x and guiding catheter 104 and implant, and the second introducer 102 can be retracted after the additional second introducer 102x is delivered and before the delivery of the guiding catheter 104 and implant, and that the additional second introducer 102x can be retracted after the guiding catheter 104 is delivered toward or into the anatomical target position and before the delivery of the implant 110. In this way a maximum space can be provided for the delivering catheter 104, or the guiding catheter 104, and in particularly when the guiding catheter 104 in an expandable catheter 104, whereupon a relatively big implant can be delivered into the anatomical target position. Previously, the diameter of the all additional introducers or catheters must have been smaller and smaller, whereupon the diameter of the last delivering catheter is particularly small, which remarkably limits also the size of the object, such as the implant, to be delivered.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims. For example the guide wire is at least partially formed from a shape memory material operable to assume an activated shape and an inactivated shape, wherein in said inactivated shape the guide wire is configured to be delivered in a straightened configuration through said first introducer and in said activated shape the guide wire is configured to take said at least a first curved shape within or near the anatomical target position. The guide wire is advantageously configured to be introduced before the implant into or towards the anatomical target position. However, it is to be noted that the guide wire should at least reach the target position before the implant.

In addition, it is to be noted that even if the implant is described in this document as an example to be delivered, also other kinds of object can be delivered according to the invention, such as medicaments, for example. Furthermore, even if the heart is described in many embodiments, it is to be understood that the heart is only an example of the anatomical target. Still, in addition it is to be noted that the implant can be rigid or flexible.

1. A medical arrangement configured to introduce an object from a distal end of the arrangement into an anatomical target position, the medical arrangement comprising a first introducer and a guide wire, wherein:
said guide wire has at least a first curved shape;
said at least first curved shape has a preformed shape capable of being delivered in a straightened configuration through said first introducer and is configured to be activated to said at least first curved shape within or near the anatomical target position;
said guide wire is configured to be introduced before the object into or towards said anatomical target position;
said object is configured to be delivered along the guide wire into said anatomical target position after the guide wire is at least partially introduced from the distal end portion of the first introducer, whereupon said object is configured to follow said guide wire and said first curved shape of the guide wire into said anatomical target position; and
said guide wire is configured to be retracted after said object has been introduced into said anatomical target position so that said object maintains the shape taken when introduced into said anatomical target position.

2. The medical arrangement of claim 1, wherein said object is configured to be delivered along the guide wire into said anatomical target position after the guide wire is at least partially introduced from the distal end portion of the first introducer and when the guide wire is activated to said at least first curved shape.

3. The medical arrangement of claim 1, further comprising a second introducer, where said second introducer is arranged to be operable between the first introducer and the guide wire.

4. The medical arrangement of claim 3, wherein the second introducer is configured to be introduced from the distal end portion of the first introducer and wherein at least a distal portion of the second introducer is configured to be introduced from the distal end portion of the first introducer before the guide wire.

5. The medical arrangement claim 3, wherein the second introducer is a catheter and wherein said first introducer is a catheter.

6. The medical arrangement of claim 3, wherein said object is configured to be delivered inside the second introducer into or towards said anatomical target position after at least the distal portion of the second introducer is introduced from the distal end portion of the first introducer.

7. The medical arrangement of claim 3, wherein said second introducer is configured to be retracted after said guide wire is introduced into said anatomical target position or before said object is introduced into said anatomical target position.

8. The medical arrangement of claim 1, further comprising a guiding catheter is arranged to be operable between the first introducer and the guide wire, and between a second introducer and the guide wire.

9. The medical arrangement of claim 8, wherein the guiding catheter is configured to be introduced from the distal end portion of the first introducer and wherein at least a distal portion of the guiding catheter is configured to be introduced from the distal end portion of the first introducer after the guide wire is introduced into or towards said anatomical target position.

10. The medical arrangement of claim 9, wherein the guiding catheter is configured to be introduced inside or from the distal end portion of the second introducer when the second introducer is first delivered to its target position and wherein said second introducer is configured to be retracted before said object is introduced into said anatomical target position.

11. The medical arrangement of claim 8, wherein the guiding catheter is a flexible catheter, which is configured to be delivered along the guide wire into or towards said anatomical target position after the guide wire is at least partially introduced from the distal end portion of the first introducer.

12. The medical arrangement of claim 8, wherein said object is configured to be delivered inside the guiding catheter into or towards said anatomical target position after at least the distal portion of the guiding catheter is introduced from the distal end portion of the first introducer or the distal end portion of the second introducer into or towards said anatomical target position.

13. The medical arrangement of claim 8, wherein said guiding catheter is configured to be retracted after the object has been introduced into said anatomical target position and wherein the object is configured to be secured to the anatomical target position after the guiding catheter is retracted.

14. The medical arrangement of claim 1, wherein the distal end of the guide wire comprises a curvature tip portion in order to prevent the distal end of the guide wire from getting tangled in to tissue.

15. The medical arrangement of claim 1, wherein the object is an implant; said implant comprises a loop shaped support portion having one or more loops or coils; said implant is adapted to support said anatomical target position upon being fully delivered; and wherein said implant comprises a hollow tubular structure or loop, stich or turn structure attached to the implant, whereupon the implant is configured to be delivered into said anatomical target position so that it travels around said guide wire.

16. A method for introducing an object into an anatomical target position, said method comprising:
delivering a guidewire in a straightened configuration through a first introducer;
at least partially introducing said guidewire from a distal end portion of said first introducer;
activating said guidewire to have at least a first curved shape within or near the anatomical target position;
delivering said object along said guidewire into said anatomical target position by causing said object to follow said guide wire and said at least first curved shape into the anatomical target position; and
retracting said guide wire after said object has been introduced into said anatomical target position so that said object maintains a shape taken when introduced into said anatomical target position.

17. (canceled)

18. The method of claim 16, further comprising introducing a second introducer from the distal end portion of the first introducer and wherein at least a distal portion of the second introducer is introduced from the distal end portion of the first introducer before the guide wire.

19. The method of claim 18, wherein said object is delivered inside the second introducer into or towards said anatomical target position after at least the distal portion of the second introducer is introduced from the distal end portion of the first introducer.

20. The method of claim 18, wherein said second introducer is retracted after said guide wire is introduced into said anatomical target position or before said object is introduced into said anatomical target position.

21. The method of claim 18, wherein a guiding catheter is introduced from the distal end portion of the first introducer and wherein at least a distal portion of the guiding catheter is introduced from the distal end portion of the first introducer after the guide wire is introduced into or towards said anatomical target position.

22. The method of claim 21, wherein the guiding catheter is introduced inside or from the distal end portion of the second introducer when the second introducer is first delivered to its target position and wherein said second introducer is retracted before said object is introduced into said anatomical target position.

23. The method of claim 21, wherein said guiding catheter is retracted after the object has been introduced into said anatomical target position and wherein the object is secured to the anatomical target position after the guiding catheter is retracted.
